SEQUENCE LISTING (4 sequences)

SEQ ID NO: 1 - DNA, 711 bp, Artificial Sequence
  Synthetic
tggaaccctttggcccagccggccacggatattttgatgacccaaactccactctccctgcctgtcagtcttggagatcaagcctccatgtcttgcagatctagtcagagccttgttcacaataatggagacacctatctgcactggtacctgcagaagccaggccagtctccaaagctcctgctccacaaagtttccaaccgcctttctggggtcccagacaggttcagtggcagtggatcagggacagatttcacactcaagatcagcagagtggagactgaggatctgggagtttatttctgctctcaaagtacacatgttccgtggacgttcggtggaggcaccaagctggaaataaaacgtggcggcggcggctccggtggtggtggatccgaggttcagctgcagcagtcaggacctggcctggtggcgccctcacagagcctgtccatcacttgcactgtctcagggttttctttaaccagttatggagtacactgggttcgccagcctccaggaaagggtctggagggactgggagtaatatgggctggtggaagcacaaattataattcggctctcatgtccagactgagcatcagcaaagacaactccaagagccaagttttcttaaaaatgaacagtctgcaaattgatgacacagccatgtattactgtgccagaagtcctacctccactatggtacgc

SEQ ID NO: 2 - protein, 237 aa, Artificial Sequence
  Synthetic
WNPLAQPATDILMTQTPLSLPVSLGDQASMSCRSSQSLVHNNGDTYLHWYLQKPGQSPKLLLHKVSNRLSGVPDRFSGSGSGTDFTLKISRVETEDLGVYFCSQSTHVPWTFGGGTKLEIKRGGGGSGGGGSEVQLQQSGPGLVAPSQSLSITCTVSGFSLTSYGVHWVRQPPGKGLEGLGVIWAGGSTNYNSALMSRLSISKDNSKSQVFLKMNSLQIDDTAMYYCARSPTSTMVR

SEQ ID NO: 3 - DNA, 702 bp, Artificial Sequence
  Synthetic
aaccctttggcccagccggccacggaaaatgtgctcacccagtctccagccaccctatctgtgactccaggagatagcgtcagtctttcctgcagggccagcaaaagtattagcaacaatctacactggtatcaacaaaaatcacatgagtctccaagacttctcatcaagtatgcttctcagtccatttctgggatcccctccaggttcagtggcagtggatcagggacagatttcactcttagtatcaacagtgtggagaccgaagattttggaatgtatttctgtcaacagagtaacagctggccttacacgttcggaggggggaccaagctggaattaaaacgtggtggtggtggttctggtggtggtggttctggcggcggcggctccggtggtggtggatccgaggttcagctccagcagtctgggggaggcttagtgaaacctggagggtccctgaaactctcctgtgcagcctctggattccctttcagtagctatgccatgtcttgggttcgccagactccggagaagaggctggagtgggtcgcaaccattactagtggtggttcttacacctactatccagacagtgtaaaggggcgattcaccatctccagagacaaagccaagaacaccctgtacctgcaaatgggcacactgaggtctgaggacacggccatgtattactgtgctaga

SEQ ID NO: 4 - protein, 234 aa, Artificial Sequence
  Synthetic
NPLAQPATENVLTQSPATLSVTPGDSVSLSCRASKSISNNLHWYQQKSHESPRLLIKYASQSISGIPSRFSGSGSGTDFTLSINSVETEDFGMYFCQQSNSWPYTFGGGTKLELKRGGGGSGGGGSGGGGSGGGGSEVQLQQSGGGLVKPGGSLKLSCAASGFPFSSYAMSWVRQTPEKRLEWVATITSGGSYTYYPDSVKGRFTISRDKAKNTLYLQMGTLRSEDTAMYYCAR

(19) United States
(12) Patent Application Publication
(10) Pub. No.: US 2008/0188007 A1
(43) Pub. Date: Aug. 7, 2008

(54) Fluorescent single chain antibody and its use in detection of analytes

(75) Inventors: K. John Morrow, Newport, KY (US); Xiaokang Deng, Cincinnati, OH (US); Robert F. Devlin, Tumbling Shoals, AR (US); Preston H. Dorsett, Memphis, TN (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: Meridian Life Science, Inc.

(21) Appl. No.: 11/702,812

(22) Filed: Feb. 6, 2007

(51) Int. Cl. G01N 33/566; C07K 16/44

(52) U.S. Cl. 436/501; 530/391.3

(57) ABSTRACT

A fluorescent single chain antibody construct is provided comprising a single chain antibody reactive with a target analyte and conjugated with a fluorophore. Also provided is a method for rapid and accurate detection of analytes using the fluorescent single chain antibody construct thereof. Further provided is a diagnostic kit for detecting pathogens using a fluorescent single chain antibody construct comprising a single chain antibody reactive with pathogen-related antigens and conjugated with a fluorophore.

### FIELD OF THE INVENTION

The present invention generally relates to analyte detection and medical diagnostics. In particular, the present invention relates to fluorescent single chain antibody constructs and method for detecting analytes using the constructs thereof. The present invention also relates to diagnostic kits for detecting infectious pathogens.

### BACKGROUND OF THE INVENTION

Analytes are chemical entities, detectable through many technologies. In the assessment of disease states, analytes are biological markers of pathogenicity. These may be toxins, enzymes or other markers produced by an invading organism, or proteins generated in response to trauma or pathology. Analytes are usually proteins, but they may also be liposaccharides, polysaccharides or nucleic acids.

The most widely used reagents for detection of proteins and related molecules are antibodies. Antibodies are complex protein molecules produced in response to the presence of foreign substances, referred to as antigens. An antibody possesses a “variable” and a “constant” region. The “variable” region is a region that compounds with the antigen and endows the antibody with specificity, enabling the antibody to bind to one of the millions of different antigenic structures that can exist. The “constant” region is a region that does not vary much from one antibody to another, although different classes of antibodies may possess differences in their “constant” regions. In addition, genetic variation adds to the variability of the “constant” region, so the name applies only in a relative sense.

A variety of molecules may behave as antigens, but generally antigens are high molecular weight substances. Antigens are usually proteins, glycoproteins, polysaccharides or lipopolysaccharides. Antibodies combine in a highly specific, lock and key fashion with their target antigens, making them ideal for analyte detection. Antibodies can be polyclonal, which are complex mixtures of many molecular types present in serum of injected animals, or they can be monoclonal, which are produced by culturing lymphocytes from injected animals (e.g. mice). In particular, the lymphocytes are isolated and fused with an appropriate mouse tumor cell line, usually originating from a myeloma (i.e., a tumor of the plasma cell). The cells are placed in culture dishes under selective conditions in which only the fused cells can proliferate. After a period of two weeks, clones of cells arise in the culture dishes, which clones are then screened and selected for best candidates. The selected clones are further evaluated and re-cloned.

Monoclonal antibodies are clonal isolates, so the entire population arising from a single cell line is identical. All the antibody molecules of the same population will react with a single antigenic site, or epitope on the analyte. In recent years, antibodies have been engineered using recombinant DNA technology, which employs the manipulation of cloned gene sequences from antibody-producing cells.

A modification in recombinant antibody construction concerns the use of only the variable sequences of the antibody. By isolating the light and heavy chain variable portions of the antibody molecule, it is possible to construct a much smaller version of the original antibody with a molecular weight of about 25,000 Daltons, rather than 180,000, as is the case for a native antibody molecule. These molecules with reduced molecular weights are modified in such a way that the light chain is connected with the heavy chain through a flexible linker of repeating sequences of the amino acids glycine and serine. Usually three glycines are followed by a serine and the sequence is repeated several times. This endows the molecule with the necessary flexibility to permit the light and heavy chains to align themselves in a configuration that forms an antibody combining site.

A variety of antibody structures are well known in the art, which can be applied to immunological diagnostic tests. In order to register the presence of a target analyte, the antibody must be coupled to a detection system. Many such systems have been invented over the last century. The simplest are immunoprecipitation assays that directly detect interactions by zones of precipitation in agar, between two wells bored at different positions in the agar. When encountering one another, the diffusing molecules form a lattice, which is visible to the naked eye as a cloudy precipitation zone.

Over the decades, as chemical synthetic technology has advanced, other detection methods employing radioisotopes, fluorescent compounds, enzymes and a myriad of other detectors have been introduced. Such methods, well known to those versed in the art, frequently employ a second antibody, which binds to the first antibody directed against a target antigen. The second antibody is directed against immunoglobulin and is coupled to a detector. Commonly used detectors include peroxidase, alkaline phosphatase, β-galactosidase, radiolabels (including iodine 131), and fluorescent compounds (used primarily for Fluorescent Activated Cell Sorting (FACS) and fluorescent microscopy).

Fluorescence polarization has been widely used in platforms similar to a radioimmune assay format to measure the presence of biological molecules (Jolley and Nasir, 2003). The concept of fluorescence polarization is almost a century old, being derived from the property of many fluors to emit light in the same direction in which it is absorbed. Ordinarily, a small fluorescent molecule emits light in all directions as it rotates in solution during the excited lifetime prior to its fluorescent emission. However, if the molecule is complexed with a larger molecule, its rotation will be slowed, and the emission will occur in roughly the same direction as the absorption; it is therefore polarized. The degree of polarization is determined by measuring the emission photons parallel and perpendicular to the plane of the excitation light and expressed in terms of polarization units (p):

$$P = \frac{Parallel Intensity - Perpendicular Intensity}{Parallel Intensity + Perpendicular Intensity}$$

Polarization unit is commonly expressed as milli-polarization units or mP. For example, a fluorescein-labeled molecule with a molecular weight of 5000 has an mP of approximately 30, however, when it becomes complexed with a specific antibody, the total molecular weight becomes approximately 165,000 and the observed mP is 180 to 200. Thus, with a specific antibody and a fluorescein-labeled probe, it would become easier to detect unlabeled antigen in a biological fluid such as serum by a competitive binding assay.

In one particular application of the fluorescence polarization technology, a low molecular weight analyte is coupled to a fluorescing molecule, and a standard curve is executed in which the coupled analyte is used to compete against non-labeled analyte in the presence of an analyte-specific antibody. As more of the non-labeled analyte is added to the mixture, the signal decreases and a curve of fluorescence polarization signal as a function of non-labeled analyte is generated. If a test sample is added to this mixture, the amount of decrease in the fluorescence polarization signal is proportional to the amount of a target molecule existing in the test sample.

In another application of the fluorescence polarization technology, the amount of specific antiserum reactive with a particular low molecular weight analyte can be measured by adding the fluorescently labeled analyte to the serum sample and then measuring the fluorescence polarization signal, which signal is proportional to the amount of the specific antiserum in the sample.

These platforms, and variations thereof, are sensitive and efficient for assaying the presence of low-molecular weight substances such as hormones. Fluorescence polarization technology has also been used to measure antigen-antibody interactions without relying on separation of unbound from bound reactants (Portmann et al. 1971; Dandliker et al. 1973; Dandliker et al. 1981; Devlin et al. 1993; Lin and Nielsen, 1997). Interactions of picomolar quantities of tracers are routine. As shown in FIG. 1, in a mixture of large and small molecules coupled to a fluorescent compound, the small molecules quickly lose their orientation and the fluorescence polarization signal associated with the small molecules declines, whereas the large molecules are not randomized as rapidly and maintain their orientation in the plane of polarized light. Also shown in FIG. 1 is that fluorescein molecules or other fluorochromes, are selectively excited by photons of polarized light with their absorption transition vectors aligned parallel to the electric vector of polarized light (along the Y axis). Small molecules rotate rapidly in solution, and quickly lose their polarization signal as they randomize (see “low” portion of the Y axis). However, if the fluorescent probe becomes complexed with a larger, slowly rotating molecule, such as an antibody, the emission is much less random and the polarization remains high (see “high” portion of the Y axis).

Klein et al. (U.S. Pat. No. 5,070,025) describes development of a fluorescence polarization assay using a fluorescently labeled peptide which competes with a target protein for an antibody reactive with both the target protein and the peptide (which contains a sequence derived from the target protein). The greater the concentration of the target protein is, the greater the suppression of the fluorescence polarization signal is. This assay has not been widely adopted since it requires production of a peptide that bears common immunoreactive sites with the protein from which it is derived, and development of an antibody reactive with both the peptide and the protein.

In another approach, Montelaro et al. (U.S. Pat. No. 6,350,574) describes an assay for detecting equine infectious anemia virus, a lentivirus related to human immunodeficiency virus type 1 (HIV-1). In particular, a series of peptides from the immunodominant gp45 protein were evaluated for their abilities to act as detectors when labeled with 5-carboxy fluorescein. In this case, serum positive equines were detected. However, this assay is not a general one and can only be applied to the specific disease in question.

Up to date, fluorescence polarization has not been used to measure the amount of high molecular weight proteins in blood, urine, feces or other biological samples. For example, it has previously been impossible to use a fluor-labeled antibody as a probe to detect antigens since the antibody itself has a molecular weight of 160,000 and an mP of 180 to 200. Although the antibody-antigen complex may have a much larger molecular weight than the antibody alone, there is not enough dynamic range available to use fluorescence polarization in an antigen detection system when the antibody molecule is carrying the label.

Thus, there is a need for effective methods for detecting high molecular weight analytes, for example, antigens. The present invention fulfills this need.

### SUMMARY OF THE INVENTION

The present invention provides a fluorescent single chain antibody construct comprising a single chain antibody reactive with a target analyte and conjugated to a fluorophore. The present invention also provides a method for detecting a high molecular weight analyte using the fluorescent single chain antibody thereof. A diagnostic kit for detecting an infectious pathogen is further provided.

In particular, the present invention is directed to a fluorescent single chain antibody construct comprising a single chain antibody and a fluorophore conjugated to the single chain antibody. The single chain antibody consists of an antigen binding region of a light chain, an antigen binding region of a heavy chain and a flexible linker connecting the two antigen binding regions.

The present invention is also directed to a method for detecting the presence of an analyte in a sample. This method includes the steps of: (a) preparing a buffered solution of the sample; (b) adding to the buffered solution a fluorescent single chain antibody construct of the present invention; (c) incubating for a period of time sufficient to permit reaction to take place between the sample and the fluorescent single chain antibody construct to produce a reaction product; and (d) measuring the degree of fluorescence polarization in the reaction product. An increase of the degree of fluorescence polarization in the reaction product as compared to degree of fluorescence polarization of the single chain antibody construct indicates the presence of the analyte in the sample.

The present invention is further directed to a method for diagnosing a pathologic condition in a subject. This method includes the steps of: (1) preparing a buffered solution of a specimen from the subject; (b) adding a fluorescent single chain antibody construct of the present invention to the buffered solution, wherein the single chain antibody is reactive with an antigen that is related to a pathogen specific for the pathologic condition; (c) incubating for a period of time sufficient to permit reaction to take place between the specimen and fluorescent single chain antibody construct to produce a reaction product; and (d) measuring the degree of fluorescence polarization in the reaction product. An increase of degree of fluorescence polarization in the reaction product compared to degree of fluorescence polarization of the single chain antibody construct is indicative of the presence of the pathogen in the specimen, which in turn is indicative of the pathologic condition in the subject.

The present invention is still further directed to a diagnostic kit for detecting the presence of an infectious pathogen, which comprises a diagnostically effective amount of a fluorescent single chain antibody construct of the present invention and a suitable container, wherein the single chain antibody is reactive with an antigen related to the pathogen.

The foregoing and other advantages of the present invention will be apparent to one of ordinary skill in the art, in view of the following detailed description of the preferred embodiment of the present invention, taken in conjunction with the accompanying drawings.

### BRIEF DESCRIPTION OF THE DRAWINGS

Features of the present invention as well as a preferred mode of use, further objectives, and advantages thereof, will best be understood by reference to the following detailed description of an illustrative embodiment when read in conjunction with the accompanying drawings, wherein:

FIG. 1 is a graph illustrating fluorescence polarization concept. X axis: time, Y axis: magnitude of fluorescence polarization signal.

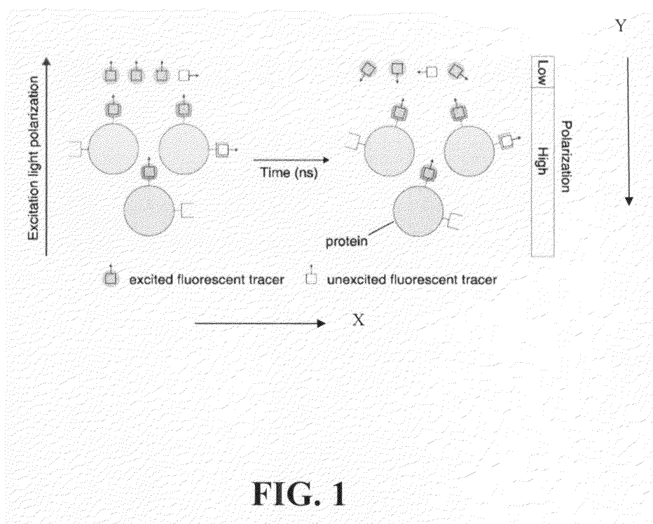

FIG. 2 is a scheme illustrating a monomeric fluorescent single chain antibody construct in accordance with the present invention.

FIG. 3 is a scheme illustrating a dimeric fluorescent single chain antibody construct in accordance with the present invention.

FIG. 4 is a half sandwich enzyme-linked immunoabsorbent assays (ELISA) demonstrating equivalency of single chain antibody (scFv) and its parent monoclonal antibody.

FIG. 5 shows performance of the scFv ToxA/B6 antibody in an ELISA assay, before and after labeling with fluorescein.

FIG. 6 shows fluorescence polarization signal (mp units, Y axis) as a function of concentration of Toxin A (X axis).

FIG. 7 shows fluorescence polarization signal (mp units, Y axis) as a function of Toxin A concentration in a spiked stool preparation.

### DETAILED DESCRIPTION OF PREFERRED EMBODIMENTS

The present invention refers to a fluorescent single chain antibody constructs as well as methods and kits for detecting the presence of an analyte in a sample based on the generation of a fluorescence polarization signal and its detection by a fluorescence polarization spectrometer. In particular, the present invention combines single chain antibodies with fluorescence polarization technology to allow for a specific and sensitive detection of the presence of an analyte in a sample.

In particular, the present invention is directed to a fluorescent single chain antibody construct comprising a single chain antibody and a fluorophore conjugated to the single chain antibody. The single chain antibody consists of an antigen binding region of a light chain, an antigen binding region of a heavy chain and a flexible linker connecting the two antigen binding regions.

Preferably, the single chain antibody is monomeric with a molecular weight of 25-30 kD, or dimeric with a molecular weight of 50-60 kD.

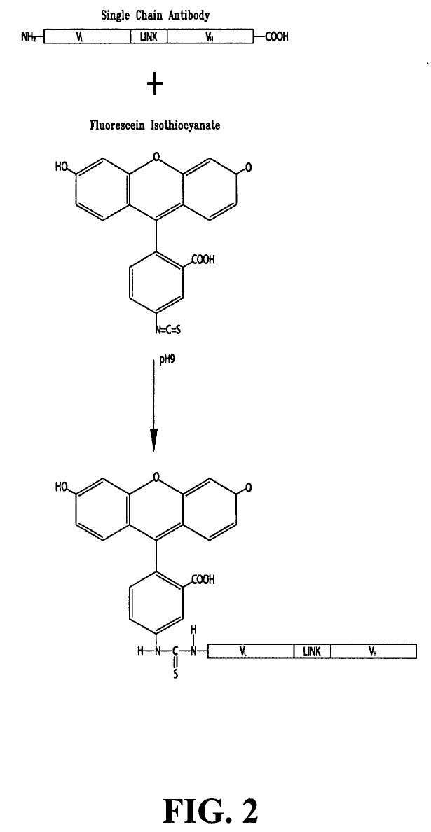

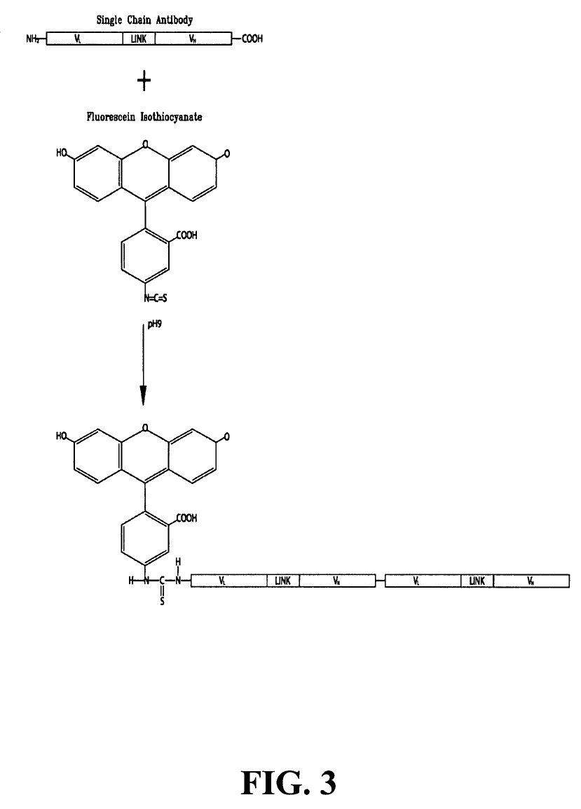

A monomeric single chain antibody is simply a variable light chain (or more suitably, an antigen binding region of a light chain), connected to a variable heavy chain (or more suitably, an antigen binding region of the heavy chain), as shown in FIG. 2. The appropriate pairing of the two chains (regions) forms a ligand binding site. Whereas a dimeric single chain antibody (scFv) is two scFvs paired in the relationship of a variable light chain from one molecule paired with a variable heavy chain from the other for the first member of the dimer pair, and vice versa for the other member of the dimmer pair so the dimer has two binding sites, as shown in FIG. 3. The association is non-covalent.

Still preferably, the fluorophore can be fluorescein isothiocyanate, phthalocyanine dye La Jolla Blue or any other suitable fluorchrome.

In a preferred embodiment, the linker connecting the light and heavy chains is a quartet of five amino acids, GLY GLY GLY GLY SER, repeated several times (this can vary, but usually 4 repeats are included). This results in a flexible linker long enough to loop back and allow the light chain to pair appropriately with the heavy chain.

The present invention is also directed to a method for detecting the presence of an analyte in a sample. This method includes the steps of: (a) preparing a buffered solution of the sample; (b) adding to the buffered solution a fluorescent single chain antibody construct of the present invention; (c) incubating for a period of time sufficient to permit reaction to take place between the sample and the fluorescent single chain antibody construct to produce a reaction product; and (d) measuring the degree of fluorescence polarization in the reaction product. An increase of the degree of fluorescence polarization in the reaction product as compared to degree of fluorescence polarization of the single chain antibody construct indicates the presence of the analyte in the sample.

Preferably, the analyte is a protein or other high molecular weight entity, such as an antigen. More preferably, the analyte is a pathogen-related antigen.

Still preferably, the single chain antibody is monomeric with a molecular weight of 25-30 kD, or dimeric with a molecular weight of 50-60 kD, and the fluorophore is fluorescein isothiocynate, phthalocyanine dye La Jolla Blue or any other suitable fluorchrome.

The present invention is further directed to a method for diagnosing a pathologic condition in a subject. This method includes the steps of: (1) preparing a buffered solution of a specimen from the subject; (b) adding a fluorescent single chain antibody construct of the present invention to the buffered solution, wherein the single chain antibody is reactive with an antigen that is related to a pathogen specific for the pathologic condition; (c) incubating for a period of time sufficient to permit reaction to take place between the specimen and fluorescent single chain antibody construct to produce a reaction product; and (d) measuring the degree of fluorescence polarization in the reaction product. An increase of degree of fluorescence polarization in the reaction product compared to degree of fluorescence polarization of the single chain antibody construct is indicative of the presence of the pathogen in the specimen, which in turn is indicative of the pathologic condition in the subject.

The present invention is still further directed to a diagnostic kit for detecting the presence of an infectious pathogen, which comprises a diagnostically effective amount of a fluorescent single chain antibody construct of the present invention and a suitable container, wherein the single chain antibody is reactive with an antigen related to the pathogen. Preferably, the single chain antibody is monomeric with a molecular weight of 25-30 kD, or dimeric with a molecular weight of 50-60 kD, and the fluorophore is fluorescein isothiocynate, phthalocyanine dye La Jolla Blue or any other suitable fluorchrome.

Single chain antibody (scFv) has been one of the most intensively studied modifications of the conventional Y-shaped antibody structure (Leath et al, 2004). This structure contains only the variable regions of the antibody molecule, or more suitably, the antigen binding regions of the antibody molecule, held together by a flexible linker consisting of repeating glycine and serine residues of variable length. Although early studies found that scFvs tended to have lower affinities than the intact antibodies from which they were derived, with sufficient effort, very high affinity scFvs can be generated (Bradbury and Marks, 2004).

In the present method, a single chain antibody is first generated against a target analyte. This is accomplished by cloning the variable antibody regions from a hybridoma cell line producing a monoclonal antibody against the target analyte, or by isolating light and heavy chain genes directly from the lymphocytes of immunized animals, or by screening genetic libraries for the desired antibody sequences using a selective method, such as phage display.

The above generated single chain antibody is then conjugated with a fluorescent compound. A variety of fluors can be employed to achieve a rigid conjugation structure, which cannot rotate upon its axis, as this would obviate the ability to produce a fluorescence polarization signal.

Once fluorescently labeled, the single chain antibody is then mixed with the sample in an appropriate buffer solution. The sample may be diluted or processed to remove particulate or opaque material before it is introduced into the buffer. After a period of time sufficient for a reaction product to be produced, fluorescence polarization signal is read by a fluorescence polarimeter. As the molecules of antibody and analyte associate, they form large complexes, which rotate much more slowly in solution compared to the molecules of the antibodies alone. follows that fluorescein-labeled scFVs should be good detectors of large antigens using fluorescence polarization technology. The scFv-antigen complex should result in a substantial increase in molecular weight and a large shift in the degree of fluorescence polarization.

Accordingly, scFv was constructed from a monoclonal antibody produced by a hybridoma as follows: The hybridoma cell lines were obtained from the Meridian Biosciences hybridoma collection. Total RNA was extracted from 5×10⁶ hybridoma cells using the TRIzol total RNA extraction protocol (Gibco BRL; Berger and Chirgwin. 1989).

5 μg total RNA was reversed transcribed in a reaction volume of 33 μl using separate reactions for light chains and heavy chains with the primers specified in Krebber, et al. (1997) according to the manufacturer's protocol (first strand cDNA synthesis kit, Pharmacia, Piscataway, N.J. Cat# 27-9261-01). The entire first-strand reaction (33 μl) was amplified to include all the cDNA of interest in the final mixture and added only the PCR primers and Taq DNA polymerase (Promega, San Luis Obispo, Calif. Cat# M1661). A cool start protocol was used. Then 40 pmol of LB and LF primer mixes was added for amplification of light chain variable domain gene (V_{L}) or 40 pmol of HB and HF primer mixes for amplification of heavy chain variable domain gene (V_{H}) (Krebber, 1997). Taq polymerase (2.5 U) was added to 33 μl of cDNA mixed with water to a final reaction volume of 100 μl. The mixes were retained on ice prior to PCR. The PCR program was as follows: denaturation at 92° C., 5 min, followed by 7 cycles of 1 min at 92° C., 30 s at 63° C., 50 s at 58° C., 1 min at 72° C., and 23 cycles of 1 min at 92° C., 30 s at 63° C., 1 min at 72° C. Subsequently, the reaction products were analyzed by running a 5 μl sample on a 1% agarose gel.

The purified PCR products of V_{L} and V_{H} sequences were prepared by a QIAquick PCR purification kit (QIAGEN Inc, Valencia, Calif., Cat# 28104). Approximately 30 ng of each V_{L} and V_{H} DNA were combined by SOE-PCR (splicing by overlap extension; LeFebver, et al, 1995). Again the cool start method was used. Taq polymerase (2.5 U) was added as before and the solution kept on ice. The following PCR program was used: denaturation at 95° C. 5 min, followed by 5 cycles of 1 min at 95° C., 30s at 63° C., 50s at 58° C., 3 min at 72° C. without primers. After adding the outer primers scback and scfor, the denaturation was repeated at 95° C. 5 min, followed by 5 cycles of 1 min at 95° C., 30s at 63° C., 50s at 58° C., 3 min at 72° C., 35 cycles of 1 min at 95° C., 1 min at 55° C., 3 min at 72° C., and also the last extension at 72° C. for 7 min was employed. 5 μl of PCR reaction mixture was analyzed by 1% agarose gel electrophoresis.

Approximately 1 μg of purified scFv fragment was digested with 30 U of SfiI in an 85-μl reaction mixture at 50° C. for 4 hours. The digested scFv fragment was recovered on a 1% agarose gel and the concentration was determined. The expression vector was prepared by digesting 10 μg of pAK400 with 60 U of SfiI in a 100-μl reaction volume at 50° C. for 4 hours. DNA was purified on 0.5% GEL and the concentration was determined. After digestion, 200 ng of the vector was ligated with 20 ng of scFv (molar ratio of vector to insert being 1:1.5) and transformed into Escherichia coli strain JM83. The transformants were plated onto an NE plate and incubated at 30° C. overnight.

Direct screening of active antibody-producing clones on 96 well microtiter plates by ELISA was carried out. Colonies were randomly picked up and inoculated into 100 μl of NE in a 96 well sterile cell culture microtiter plate, and the clones were incubated overnight at room temperature with agitation. Next morning glycerol was added into each well to the final concentration of 15% and mixed well. This plate was kept frozen to serve as master plate. For soluble scFv production in the microtiter plate format, a second 96-well microtiter plate with 100 μl NE in each well was prepared. It was inoculated with 10 μl of cells from the master plate into the 100 μl NE in the duplicated position. The second plate was grown for two hours at room temperature with agitation and another 100 of μl NE was added to each well. Then IPTG was introduced to the culture with a final concentration 0.5 mM. The plate was grown overnight at the room temperature with agitation to induce soluble scFv expression.

After induction, the plate was frozen at −80° C. for one hour, and then thawed thoroughly at 37° C. Then 100 μl lysozyme solution was added to each well to a final concentration of 1 mg/ml. The wells were thoroughly mixed by pipetting up and down, and the plate was incubated at 37° C. for 30 minutes with an additional incubation at room temperature for 20 minutes.

The lysates from each well were screened for activity. The cell lysates from each well in the second plate were transferred to a duplicate position on a 96 well high-binding plate pre-coated with the target antigen at 1 μg/well. Another plate was coated with blank PBS as a negative control. The plates were incubated at 37° C. for 1 hour followed by washing with PBST 4 times. A secondary detection antibody anti-HIS tag HRP conjugation was used to detect the scFv. The conjugates were diluted at 1:2000 in 5% milk in PBS and then added to the plate in an amount of 100 μl/well and incubated at 37° C. for 1 hour. The plate was washed thoroughly 8 times in PBS and developed with the substrate TMB at room temperature for 15 minutes. The plate was then stopped from further developing with sulfuric acid and read at OD_{450/630}.

The clones with the highest anti-TXA activities were inoculated into 50 ml of NE and grown overnight at room temperature at 250 RPM. The following morning they were transferred into 350 ml NE and grown at room temperature to an OD₅₅₀ of 0.6. Protein expression was induced by adding 0.25 mM IPTG overnight at 20° C. at 200 RPM. Following induction the cells were collected by centrifugation at 4° C. for 5 minutes at 5000 g. The supernatant was discarded and the pellet frozen at −20° C. for 1 hour. The pellet was then thawed thoroughly at room temperature, and 24 ml of lysis buffer was added with the final lysozyme concentration of 1 mg/ml to resuspend pellet. The resuspension solution was incubated at room temperature for 10 minutes, added with 960 μl of 100 mM MgCl₂ and 480 μl of 1 mg/ml DNAse I, and further incubated at room temperature for 10 more minutes. The lysates were centrifuged 40 minutes at 4° C. at 15,000 g; and the supernatant containing the soluble scFvs was retained.

To purify the scFvs, the target antigen were coupled to affigel 10 to form the affinity column, the scFv sample was applied to the column to allow binding of the scFvs to the target antigen. The non-specific materials were washed off, and the scFv were then eluted off. Purified scFvs were then used for fluorescein isothiocyanate (FITC) labeling.

FITC Labeling of scFvs

1.5 ml of scFv WB10 solution containing 31 μg/ml of the scFvs in phosphate buffer and 300 mM imidazole was placed in Centricon 10 tube (Amicon 4205, Lot# MA297) and centrifuged in Sorvall RT 6000 at 4° C. and 2300 RPM to concentrate and to remove the 300 mM imidazole. After discarding the filtrate, 1.5 ml carbonate buffer was added, followed by re-concentrating. The process of discarding, adding and re-concentrating was repeated three times. Afterwards, the final filtrate was discarded and the retention was diluted by adding 1.5 ml carbonate buffer. The Centricon tube was then covered with parafilm and stored for 72 hours at 4° C. The sample was concentrated again in Sorvall RT 6000 at 4° C. and 2300 RPM for two times. The retention was collected per Centricon instructions and adjusted to 1 ml with carbonate buffer having a pH of 9.5 (VAI 8484, Lot# 020426M). Such retention solution contained scFv WB10 ready for labeling.

A FITC stock solution was prepared by adding 3.85 ml of acetone (Fisher ACS grade, VAI 2366, Lot# 7010138) to 3.85 mg FITC (VAI 2576, Lot# 1010054). The resulting FITC solution had a concentration of 1 mg/ml in acetone solution, and subsequently covered with foil for storage.

To label the scFv WB10, 6 μl of the FITC stock solution was added to the retention solution containing scFv WB10 in a molar ratio of 10:1, covered and vortexed, followed by storing at 4° C. for overnight. Labeling reaction began thereafter. In the meantime, a Centricon 10 tube with 1 ml carbonate buffer at pH 9.5 on the membrane was stored at 4° C. for overnight.

1 ml of FITC-scFv WB10 reaction mixture was then added to the 1 ml carbonate buffer in the Centricon 10 tube along with a few extra drops of buffer to make a total volume of 2.25 ml. The tube was centrifuged as described before to concentrate for 1 hr at 4° C.

After concentration, the yellow filtrate was labeled with “First Filtrate.” About 2 ml of buffer was added to the retention (i.e., reaction mixture) and centrifuged again to wash out un-reacted FITC. Filtrate containing the un-reacted FITC was collected and labeled with “Wash 1”. Such process was repeated four more times. Filtrates that contained the un-reacted FITC were labeled with “Wash 2”, “Wash 3”, “Wash 4” and “Wash 5”, respectively. The retention, First Filtrate and Wash 1-Wash 5 were further analyzed.

### ELISA Evaluation of Antibody Activity

To determine the linear range of reactivity of the scFvs, constant amounts of antigen were coated on high binding microtiter plates, and then serial half and half dilutions of antibodies were added to the plate to detect toxin A. The secondary anti-mouse HRP was used to detect the monoclonal antibody and an anti-HIS HRP was used to detect the scFvs. After applying the substrate TMB, the plates were read at OD_{450/630}. A subsequent ELISA was used to determine the detection limits of the antibodies. An optimized working dilution was determined from the first ELISA by choosing a dilution below the upper limit of the linear range. Antibodies were applied to a plate pre-coated with a serial 50/50 dilution of antigen. Then the secondary antibodies as above were used to detect the primary antibodies and the plates were finished as above.

Labeling of Purified Anti-Human IgG Single Chain Antibodies with FITC

A single chain antibody was suspended in 300 ng/μl in PBS. A buffer exchange to 20 mM sodium phosphate was accomplished by adding 100 μl of the scFv antibody solution to 2.4 ml of 20 mM sodium phosphate, pH 8.4 and centrifuging in a Centricon 10 Concentrator for 1 hour at 2600 RPM in a Sorvall RT6000 centrifuge. The liquid was concentrated to 0.5 ml, and the procedure was repeated three more times. The Centricon collection tube was placed on a filter unit and the single chain antibody was collected by further centrifugation. FITC (0.2 μg/μl) was added to the scFv solution (0.7 ml) and the reaction mixture was incubated at 4° C. overnight. The mixture was transferred to a Centricon 10 tube and the reaction vial was rinsed with 1 ml of PBS, which was added to the Centricon 10 tube. The tube was centrifuged and the filtrate was washed 4 times with buffer.

Fluorescein Labeling of Anti-TXA scFv B6

The scFv B6 was labeled with FITC as follows: 500 ul of the scFv B6 at 0.19 mg/ml was exchange into 50 mM borate buffer, pH 8.5 by dialysis. FITC was dissolved in DMF at 10 mg/ml. A 24 fold molar excess of FITC was added to the tube, mixed well and incubated at room temperature for 1 hour. The excess FITC was removed by extensive dialysis against PBS.

The following examples are given for the purpose of illustrating various embodiments of the invention and are not meant to limit the present invention in any fashion.

### EXAMPLE 1

### Evaluation of Fluorescence Polarization Technology Using Single Chain Antibody (ScFv) Directed Against Human IgG

A scFv was generated from a hybridoma that produces a monoclonal antibody reactive with human IgG, as described above. Subsequent to the isolation and partial purification of the scFv, human IgG was immobilized on a Protein A column and the purified recombinant anti-IgG scFv antibody was allowed to bind to the immobilized human IgG. Then FITC was conjugated to the scFv in situ to protect the active site of the scFv. The bound and FITC-conjugated scFv was eluted from the column using a low pH buffer, and used as a probe to detect IgG in solution. The results yielded from the 10 minute fluorescence polarization experiment were: the labeled scFv alone exhibited an fluorescence polarization value of 75 mP; and the labeled scFv in the presence of human IgG exhibited an fluorescence polarization value of 198. The reduction of rotation results in a larger proportion of the molecules remaining oriented upon emission in the same direction as the molecules were in when they absorbed the polarized light, thus generating a stronger fluorescence polarization signal.

The present detection method is simple to operate, as it does not require any chemical reactions to take place in order for a fluorescence polarization signal to be generated or un-reacted antibody-antigen complex be removed from the solution. The present detection method is also fast in achieving results, as equilibration takes place in a matter of minutes, depending on the affinity of the antibody. Modem fluorescence polarization spectrometers can be interfaced with a computer, so large numbers of patients' data can be easily tracked and downloaded into a diagnostic laboratory's data base. As such, the present method can be employed in a clinical diagnostic facility or in a point of care situation.

In general, the present detection method typically comprises the following steps: constructing single chain antibodies (scFvs); coupling the scFvs to appropriate fluors; evaluating performance on pure antigen samples using a fluorescence polarimeter; and finally detecting the presence of the target antigen using clinical patient material, which steps are outlined below.

### Preparation of Single Chain Antibodies

Single chain antibodies (scFvs) were generated from hybridomas (Krebber et al. 1997). Two examples were scFv B6 (anti-Clostridium difficile Toxin A; SEQ ID NO:1 for DNA sequence, SEQ ID NO:2 for amino acid sequence) and scFv WB10 (anti-Toxin B; SEQ ID NO:3 for DNA sequence, SEQ ID NO:4 for amino acid sequence). These scFvs typically have a low molecular weight of in the range of 29 kDa. Since these scFvs retain an antigen binding site, it

These values clearly establish a specific reactivity between the scFv and the antigen. The scFv used in the present study was shown to be in the monomeric form when analysed by gel electrophoresis and western blotting.

### EXAMPLE 2

Evaluation of Fluorescence Polarization Technology Using ScFv Directed against Clostridium difficile Toxin A

As established in above Example 1, the fluorescence polarization (FP) technology is a viable option for the detection of protein antigens through the use of a monomeric scFv. The FP-scFv technology was next demonstrated in a system that would make use of antigen-antibody combinations applicable to Meridian's diagnostic technology needs. In this study, Clostridium difficile, a Gram-positive, spore-forming anaerobic bacillus was to be detected. This organism was first described in 1935 (Hall and O'Toole), but it was not associated with antibiotic-related diarrhea until the late 1970s (Bartlett, et al, 1977; Bartlett et al, 1978; Larsen et al, 1977). Clostridium difficile infection can lead to severe complications and currently is the most common cause of nosocomial diarrhea, often adding up to 2 weeks to the length of the hospitalization, at an additional cost of $6,000-$10,000 per case (Anand, et al, 1994; Djuretic, et al, 1996; Kelly and La Mont 1998; Ruben et al, 1995; Ryu and Nam, 2000; Sastry et al, 1989; Skerra and Pluckthun, 1988).

Clostridium difficile produces two exotoxins responsible for the pathogenesis of this diarrhea, toxin A and toxin B (Pothoulakis and LaMont. 1993). Enzyme-linked immunoabsorbent assays (ELISA) have been developed to detect toxin A and/or toxin B in stool specimens and have a sensitivity of 71% to 94% and a specificity of 92.5% to 98% (Rath et al, 1988). Because of the rapidity of testing and ease of performance, ELISAs and lateral flow assays for toxins A and B are now used most frequently by clinical laboratories for diagnosis of Clostridium difficile infection.

To test efficacy and viability of FP-scFv technology in detecting Clostridium difficile, an scFv was engineered from the hybridoma PCG4 reactive with Clostridium difficile Toxin A, as described above.

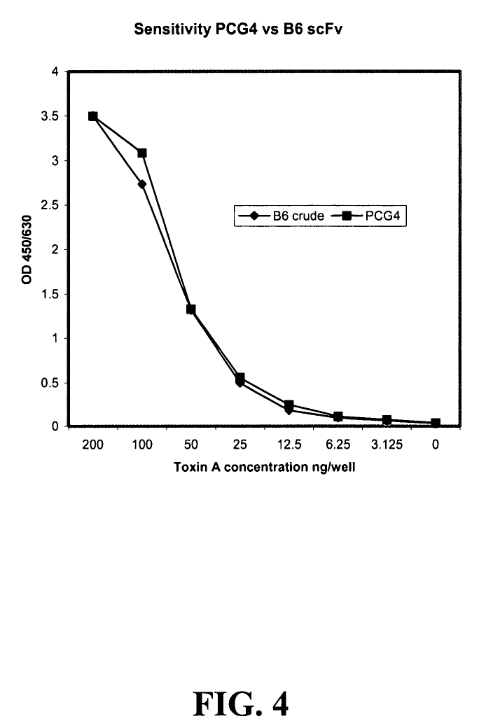

One of these (B6) gave the highest reading and was used for the validation of the fluorescence polarization platform. When comparing sensitivity, B6 and the parent monoclonal antibody had almost identical performances (FIG. 4). In this assay, wells of an Immulonmicrotiter plate were coated overnight with purified Toxin A antigen dissolved in phosphate buffered saline (PBS) buffer at the concentrations indicated. The wells were then blocked for one hour with 5% dried milk in PBS, incubated for 1 hour with the antibodies, followed by incubation with a second detector antibody coupled to horseradish peroxidase (HRP) at a 1/2000 dilution. The results suggest that the cloning procedure did not result in a loss of sensitivity. This was a fortuitous outcome, as it is common for scFvs to give up some of their binding capability when a portion of the molecule is cloned and evaluated.

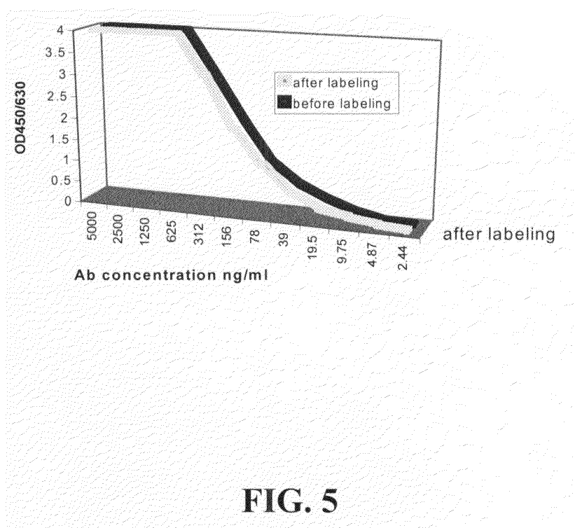

The antibody was then labeled with FITC as described above. The binding activity of the antibody was measured by a standard ELISA procedure before and after labeling with the FITC. The data shows that there was no loss in activity (FIG. 5). Assays were performed by diluting the samples and scFvs in PBS buffer. In detail, Immunlon plate was coated with 200 ng/well of Toxin A protein, blocked with 5% milk in PBS and the antibody preparations were diluted as indicated. The plates were then reacted with an anti-kappa monoclonal antibody labeled with HRP.

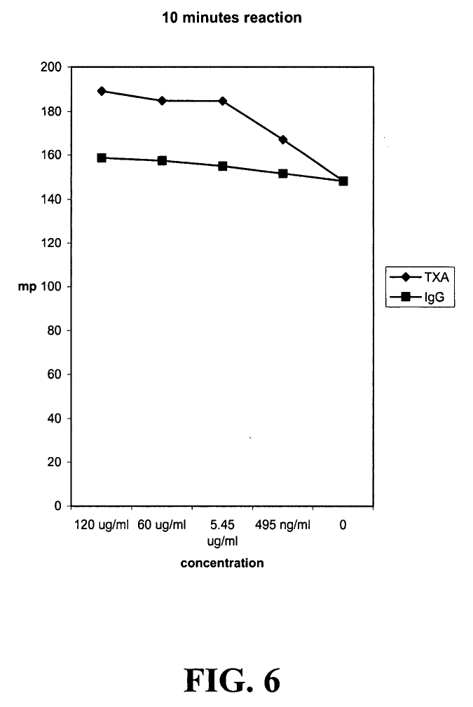

A number of conditions were investigated in order to optimize the assay procedure. Three different concentrations of the antibody, corresponding to 1, 2, and 3 μg/ml, were evaluated in the fluorescence polarization assay. In each case, a control antigen (IgG) was employed. The fluorescence polarization signal is essentially flat over the entire range of concentrations in the control, indicating that the assay procedure is specific for the antigen targeted by the scFv. The degree of fluorescence polarization ranged over 40 mp from the highest to the zero concentration of the antigen, and such method was capable of detecting the toxin with a concentration of as low as 495 ng/ml (FIG. 6). In this assay, the toxin or a control antigen (human IgG) was diluted as indicated in PBS. The scFv-fluorescein conjugate was added and the samples were read after a 10 minute interval in the fluorescence polarization reader. Longer incubation intervals (20 and 50 minutes) did not significantly alter the response.

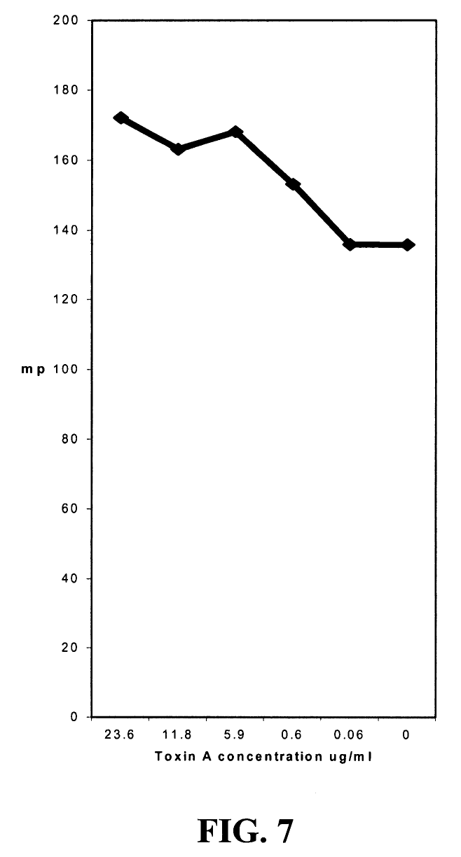

A critical test of the assay procedure is its ability to perform under real life conditions. The Meridian Bioscience products for Clostridium difficile diagnosis are stool-based assays, so an evaluation of the fluorescence polarization platform was carried out using a Clostridium difficile-negative stool sample, spiked with a range of concentrations of purified toxin A. Accordingly, the stool specimen, diluted in buffer, was mixed with the fluorescinated scFv and a range of toxin A concentrations. The samples were centrifuged and read in the fluorescence polarization instrument. As can be observed from FIG. 7, the presence of a stool sample had no effect on the sensitivity of the assay, as the assay was capable of detecting the toxin with a concentration of as low as 0.5 μg/ml. In this assay, samples were mixed and centrifuged to remove opaque material. They were incubated for 10, 30 and 50 minutes and then read in a fluorescence polarization reader.

It will be obvious to one of ordinary skill in the art that various changes may be made without departing from the scope of the invention, which is not to be considered limited to what is described in the specification.

1. A fluorescent single chain antibody construct comprising:
(a) a single chain antibody consisting of an antigen binding region of a light chain, an antigen binding region of a heavy chain and a flexible linker connecting said antigen binding region of said light chain and said antigen binding region of said heavy chain; and
(b) a fluorophore conjugated to said single chain antibody.

2. The fluorescent single chain antibody construct of claim 1, wherein said single chain antibody is monomeric with a molecular weight of 25-30 kD, or dimeric with a molecular weight of 50-60 kD.

3. The fluorescent single chain antibody construct of claim 1, wherein said fluorophore is fluorescein isothiocyanate, phthalocyanine dye La Jolla Blue or any other suitable fluorchrome.

4. A method for detecting the presence of an analyte in a sample, comprising the steps of:
(a) preparing a buffered solution of said sample;
(b) adding to said buffered solution a fluorescent single chain antibody construct according to claim 1, wherein said construct is reactive with said analyte;
(c) incubating for a period of time sufficient to permit reaction to take place between said sample and said fluorescent single chain antibody construct to produce a reaction product; and
(d) measuring the degree of fluorescence polarization in said reaction product; wherein an increase of said degree of fluorescence polarization in said reaction product as compared to degree of fluorescence polarization of said single chain antibody construct is indicative of the presence of said analyte in said sample.

5. The method of claim 4, wherein said analyte is a protein or other high molecular weight entity.

6. The method of claim 5, wherein said analyte is an antigen.

7. The method of claim 6, wherein said analyte is a pathogen-related antigen.

8. The method of claim 4, wherein said single chain antibody is monomeric with a molecular weight of 25-30 kD, or dimeric with a molecular weight of 50-60 kD.

9. The method of claim 4, wherein said fluorophore is fluorescein isothiocynate, phthalocyanine dye La Jolla Blue or any other suitable fluorchrome.

10. The method of claim 4, where said buffer solution is a phosphate buffered saline solution.

11. A method for diagnosing a pathologic condition in a subject, comprising the steps of:
(a) preparing a buffered solution of a specimen from said subject;
(b) adding to said buffered solution a fluorescent single chain antibody construct according to claim 1, wherein said construct is reactive with an antigen, and said antigen is related to a pathogen specific for said pathologic condition;
(c) incubating for a period of time sufficient to permit reaction to take place between said specimen and fluorescent single chain antibody construct to produce a reaction product; and
(d) measuring the degree of fluorescence polarization in said reaction product; wherein an increase of said degree of fluorescence polarization in said reaction product as compared to degree of fluorescence polarization of said single chain antibody construct is indicative of the presence of said pathogen in said specimen, which is thereby indicative of said pathologic condition in said subject.

12. The method of claim 11, wherein said single chain antibody is monomeric with a molecular weight of 25-30 kD, or dimeric with a molecular weight of 50-60 kD.

13. The method of claim 11, wherein the fluorophore is fluorescein isothiocynate, phthalocyanine dye La Jolla Blue or any other suitable fluorchrome.

14. The method of claim 11, where the buffer solution is a phosphate buffered saline solution.

15. A diagnostic kit for detecting the presence of an infectious pathogen, comprising:
(a) a diagnostically effective amount of a fluorescent single chain antibody construct according to claim 1; and
(b) a suitable container,
wherein said fluorescent single chain antibody construct is reactive with an antigen related to said pathogen.

16. The diagnostic kit of claim 15, wherein said antigen is a protein or other high molecular weight entity.

17. The diagnostic kit of claim 15, wherein said single chain antibody is monomeric with a molecular weight of 25-30 kD, or dimeric with a molecular weight of 50-60 kD.

18. The diagnostic kit of claim 15, wherein the fluorophore is fluorescein isothiocynate, phthalocyanine dye La Jolla Blue or any other suitable fluorchrome.

19. The fluorescent single chain antibody construct of claim 2, wherein said single chain antibody comprises a sequence as set forth in SEQ ID NO:2 or SEQ ID NO:4.

20. The fluorescent single chain antibody construct of claim 19, wherein said single chain antibody is encoded by a sequence as set forth in SEQ ID NO:1 or SEQ ID NO:3.

21. The method of claim 8, wherein said single chain antibody comprises a sequence as set forth in SEQ ID NO:2 or SEQ ID NO:4.

22. The method of claim 21, wherein said single chain antibody is encoded by a sequence as set forth in SEQ ID NO:1 or SEQ ID NO:3.

23. The method of claim 12, wherein said single chain antibody comprises a sequence as set forth in SEQ ID NO:2 or SEQ ID NO:4.

24. The method of claim 23, wherein said single chain antibody is encoded by a sequence as set forth in SEQ ID NO:1 or SEQ ID NO:3.

25. The diagnostic kit of claim 17, wherein said single chain antibody comprises a sequence as set forth in SEQ ID NO:2 or SEQ ID NO:4.

26. The diagnostic kit of claim 25, wherein said single chain antibody is encoded by a sequence as set forth in SEQ ID NO:1 or SEQ ID NO:3.

27. A single chain antibody comprising a sequence as set forth in SEQ ID NO:2 or SEQ ID NO:4.

28. The single chain antibody of claim 27, wherein said single chain antibody is encoded by a sequence as set forth in SEQ ID NO:1 or SEQ ID NO:3.
